(19) United States
(12) Patent Application Publication
(10) Pub. No.: US 2023/0165623 A1
(43) Pub. Date: Jun. 1, 2023

(54) SYSTEMS, METHODS, AND CATHETERS FOR ENDOVASCULAR TREATMENT OF A BLOOD VESSEL

(71) Applicant: TVA MEDICAL, INC., Franklin Lakes, NJ (US)

(72) Inventors: OLADIPO PETER AKERELE-ALE, Austin, TX (US); ASEEM SINGH, Tempe, AZ (US)

(73) Assignee: TVA MEDICAL, INC., Franklin Lakes, NJ (US)

(21) Appl. No.: 17/997,470

(22) PCT Filed: Apr. 28, 2020
(86) PCT No.: PCT/US2020/030178

§ 371 Date: Oct. 28, 2022

(51) Int. Cl. A61B 18/10; A61B 34/00

(52) U.S. Cl. CPC A61B 18/10; A61B 34/73; A61B 2018/00404

(57) ABSTRACT

Systems for forming a fistula between two blood vessels are provided. In embodiments, the system may include a catheter having a housing and a treatment portion coupled to the housing. The treatment portion may include a thermoelectric generator comprising an exposed surface exposed outside of the housing and a concealed surface opposite and electrically connected to the exposed surface. The thermoelectric generator may be configured to produce a temperature differential between the exposed surface and the concealed surface when an electric current is applied to one of the exposed surface and the concealed surface thereby producing the temperature differential between the exposed surface and the concealed surface such that the exposed surface is heated to a temperature greater than the concealed surface to weld the two blood vessels together.

### TECHNICAL FIELD

The present specification generally relates to systems, methods, and catheters for treatment of a blood vessel and, more specifically, systems, methods, and catheters for endovascular treatment of a blood vessel.

### BACKGROUND

Endovascular treatments are used to treat various blood vessel disorders from within the blood vessel. Endovascular treatments may include, but are not limited to, endovascular arteriovenous fistula (endoAVF) formations, arteriovenous (AV) treatments, and peripheral arterial disease (PAD) treatments. Treatments such as endovascular fistula formation may include invasive procedures where the patient’s vein is attached to his or her artery via sutures.

### SUMMARY

One challenging aspect of endovascular treatment involves providing a fistula formation treatment that is minimally invasive with precise and modulated temperature control. Accordingly, a need exists for alternative systems, methods, and catheters for endovascular treatment of a blood vessel that improve fistula formation.

The present embodiments address the above referenced problems. In particular, the present disclosure is directed to systems, methods, and catheters for delivery of treatments to a blood vessel (e.g., fistula formation) using one or more catheters. In embodiments, the presently-disclosed catheters and catheter systems may provide endovascular fistula formation treatment that is minimally invasive with precise and modulated temperature control.

According to one aspect of the present disclosure, a system for forming a fistula between two blood vessels is provided. The system may include a catheter having a housing and a treatment portion coupled to the housing. The treatment portion may include a thermoelectric generator comprising an exposed surface exposed outside of the housing and a concealed surface opposite and electrically connected to the exposed surface. The thermoelectric generator may be configured to produce a temperature differential between the exposed surface and the concealed surface when an electric current is applied to one of the exposed surface and the concealed surface thereby producing the temperature differential between the exposed surface and the concealed surface to weld the two blood vessels together.

A second aspect may include the first aspect, further comprising an energy source coupled to the thermoelectric generator.

A third aspect may include any preceding aspect, wherein the energy source is a handheld energy source coupled to the thermoelectric generator.

A fourth aspect may include any preceding aspect, wherein the thermoelectric generator comprises at least one n-type semiconductor and at least one p-type semiconductor disposed between and electrically connected to the exposed surface and the concealed surface.

A fifth aspect may include any preceding aspect, wherein the thermoelectric generator comprises a thermoelectric material.

A sixth aspect may include any preceding aspect, further comprising one or more location indicators configured to provide location information of the treatment portion of the catheter as it is advanced through a subject.

A seventh aspect may include any preceding aspect, further comprising one or more biasing mechanisms configured to contact a wall of a blood vessel to bias the treatment portion of the catheter into contact with the wall of the blood vessel.

According to an eighth aspect of the present disclosure, a system for forming a fistula between two blood vessels is provided. The system may include a first catheter configured to be received in a first vessel and a second catheter configured to be received in a second vessel adjacent to the first vessel. The first catheter may include a housing and a treatment portion coupled to the housing, wherein the treatment portion comprises a thermoelectric generator comprising an exposed surface exposed outside of the housing and a concealed surface opposite and electrically connected to the exposed surface. The thermoelectric generator may be configured to produce a temperature differential between the exposed surface and the concealed surface when an electric current is applied to one of the exposed surface and the concealed surface thereby producing the temperature differential between the exposed surface and the concealed surface to weld the two blood vessels together.

A ninth aspect may include the eighth aspect, further comprising an energy source coupled to the thermoelectric generator.

A tenth aspect may include any of the eighth through ninth aspects, wherein the energy source is a handheld energy source coupled to the thermoelectric generator.

A eleventh aspect may include any of the eighth through tenth aspects, wherein the thermoelectric generator comprises at least one n-type semiconductor and at least one p-type semiconductor disposed between and electrically connected to the exposed surface and the concealed surface.

A twelfth aspect may include any of the eighth through eleventh aspects, wherein the second catheter comprises a second housing and a second treatment portion coupled to the second housing, wherein the second treatment portion comprises a second thermoelectric generator.

A thirteenth aspect may include any of the eighth through twelfth aspects, wherein the first catheter, the second catheter, or both comprise one or more location indicators configured to provide location information of the first catheter, the second catheter, or both as it is advanced through a subject.

A fourteenth aspect may include any of the eighth through thirteenth aspects, wherein the thermoelectric generator comprises at least one n-type semiconductor and at least one p-type semiconductor disposed between and electrically connected to the exposed surface and the concealed surface.

According to a fifteenth aspect of the present disclosure, a method of forming a fistula is provided. The method may include advancing a first catheter into a first vessel and advancing a second catheter into a second vessel. The second vessel may be adjacent to the first vessel. The first catheter may include a housing and a treatment portion coupled to the housing, where the treatment portion includes a thermoelectric generator comprising an exposed surface exposed outside of the housing and a concealed surface opposite and electrically connected to the exposed surface. The second catheter may include a second treatment portion. The thermoelectric generator may be configured to produce a temperature differential between the exposed surface and the concealed surface when an electric current is applied to one of the exposed surface and the concealed surface thereby producing the temperature differential between the exposed surface and the concealed surface wherein the exposed surface is heated to a temperature greater than the concealed surface to weld the first blood vessel to the second blood vessel.

A sixteenth aspect may include the fifteenth aspect, further comprising applying an electric current to the thermoelectric generator to produce the temperature differential.

A seventeenth aspect may include any of the fifteenth through sixteenth aspects, further comprising reversing the electric current to produce a temperature differential between the exposed surface and the concealed surface such that the exposed surface is cooled to a temperature lesser than the concealed surface.

An eighteenth aspect may include any of the fifteenth through seventeenth aspects, where the first catheter further comprises a first magnet, and the second catheter comprises a second magnet, and where the magnets are configured to bring the first catheter and the second catheter in closer approximation.

A nineteenth aspect may include the eighteenth aspect, further comprising aligning the first magnet and the second magnet to align the treatment portion of the first catheter and the second treatment portion second catheter.

A twentieth aspect may include any of the fifteenth through nineteenth aspects, further comprising applying an electric current to the thermoelectric generator to produce the temperature differential after aligning the first magnet and the second magnet.

These and additional features provided by the embodiments described herein will be more fully understood in view of the following detailed description, in conjunction with the drawings.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, where like structure is indicated with like reference numerals and in which:

FIG. 1 is an illustrative depiction of the vascular anatomy of an arm in which an endovascular treatment may be delivered, according to one or more embodiments shown and described herein;

FIG. 2 depicts a two catheter system, according to one or more embodiments shown and described herein;

FIG. 3 depicts a single catheter system, according to one or more embodiments shown and described herein;

FIG. 4A depicts a top view of a thermoelectric generator, according to one or more embodiments shown and described herein;

FIG. 4B depicts a side view of a thermoelectric generator, according to one or more embodiments shown and described herein;

FIG. 5A depicts a two catheter system within adjacent vessels, according to one or more embodiments shown and described herein;

FIG. 5B depicts a two catheter system within adhered vessels, according to one or more embodiments shown and described herein;

FIG. 6A depicts a cross-sectional view of adhered vessels, according to one or more embodiments shown and described herein;

FIG. 6B depicts a cross-sectional view of adhered vessels, according to one or more embodiments shown and described herein;

FIG. 7A depicts a cross-sectional view of adhered vessels comprising a fistula, according to one or more embodiments shown and described herein;

FIG. 7B depicts a cross-sectional view of adhered vessels comprising a fistula, according to one or more embodiments shown and described herein; and

FIG. 8 depicts communication between various modules within a system for endovascular treatment of a blood vessel, according to one or more embodiments shown and described herein.

### DETAILED DESCRIPTION

Embodiments as described herein are directed the systems, methods, and catheters for endovascular treatment of a blood vessel. Endovascular treatments may include, but are not limited to, fistula formation, vessel occlusion, angioplasty, thrombectomy, atherectomy, crossing, drug coated balloon angioplasty, stenting (uncovered and covered), lytic therapy. Accordingly, while various embodiments are directed to fistula formation between two blood vessels, other vascular treatments are contemplated and possible.

Arteriovenous (AV) fistula creation is a surgical procedure used to, for example, create an access site for hemodialysis patients. Arteriovenous fistula surgeries are invasive procedures where the patient’s vein is attached to his or her artery via sutures. Various factors can restrict the size of the patient population who have the ability to receive AV fistula treatment. Such factors may include, but are not limited to, the health of the patient and the strength of the patient’s vascular structure. These factors influence the viability of the AV fistula procedure’s success. A secondary procedure option may include AV graft placement.

Using two catheters to form a fistula or otherwise provide a treatment (e.g., advancing a wire from one blood vessel to another) has been described in U.S. Pat. No 9,017,323, entitled “Devices and Methods for Forming Fistula,” filed Nov. 16, 2011, hereby incorporated by reference in its entirety; U.S. Pat. No 9,486,276, entitled “Devices and Methods for Fistula Formation,” filed Oct. 11, 2013, hereby incorporated by reference in its entirety; U.S. Pat. Application Serial No. 14/214,503, entitled Fistula Formation Devices and Methods Therefor,” filed Mar. 14, 2014, hereby incorporated by reference in its entirety; U.S. Pat. Application Serial No. 14/657,997, filed Mar. 13, 2015, hereby incorporated by reference in its entirety; U.S. Pat. Application Serial No. 15/019,962, entitled “Methods for Treating Hypertension,” filed Feb. 9, 2016, hereby incorporated by reference in its entirety; U.S. Pat. Application Serial No. 15/406,755, entitled “Devices and Methods for Forming a Fistula,” filed Jan. 15, 2017, hereby incorporated by reference in its entirety; U.S. Pat. Application Serial No. 15/406,743, entitled “Systems and Methods for Increasing Blood Flow,” filed Jan. 15, 2017, hereby incorporated by reference in its entirety; U.S. Pat. Application Serial No. 16/024,241, entitled “Systems and Methods for Adhering Vessels,” filed Jun. 29, 2018, hereby incorporated by reference in its entirety; and U.S. Pat. Application Serial No 16/024,345, entitled “Devices and Methods for Advancing a Wire,” filed Jun. 29, 2018, hereby incorporated by reference in its entirety.

However, some common complications associated with AV fistula surgeries result from neointimal hyperplasia growth at the anastomosis of the fistula. Neointimal hyperplasia refers to vascular remodeling, resulting from proliferation and migration of vascular smooth muscle cells in the tunica intima layer, which leads to vascular wall thickening and the gradual loss of luminal patency. Ultimately, neointimal hyperplasia may lead to the return of vascular insufficiency symptoms. Additional complications arise from changes in the blood flow and the pressure gradient in the fistula.

Moreover, complications arise from the failure to provide controllable, ablative or cutting thermal energy to an intended tissue site (the “target tissue”). For example, imprecise temperature control may cause thermal energy to dissipate into the tissue surrounding the target tissue, which may cause damage to the tissue. Accordingly, the present embodiments, directed to systems, methods, and catheters for delivery of treatments (e.g., fistula formation), incorporate thermoelectric generators may provide more precise and targeted application of thermal energy for fistula formation. These and additional features will be discussed in greater detail below.

Thermoelectric generators, which will be described subsequently in more detail, are solid state devices that may generate electrical energy from a temperature differential or provide a temperature differential from electrical power. By requiring no moving parts to create the temperature differential, thermoelectric generators may reduce the complexity of two catheter systems for delivery thermal energy. Additionally, because thermoelectric generators may not require any fluids for fuel or cooling, they are non-orientation dependent to produce the temperature differential, which may be particularly advantageous during endovascular treatments within the tortuous vasculature of a subject. Therefore, the catheters, catheter systems, and methods of the present disclosure may provide endovascular fistula formation treatment that is minimally invasive while providing precise and modulated temperature control. Additional embodiments may be directed to single catheter systems that may further reduce complexity of the two catheter systems. The figures generally depict various systems, methods, and devices that incorporate thermoelectric generators used for fistula formation.

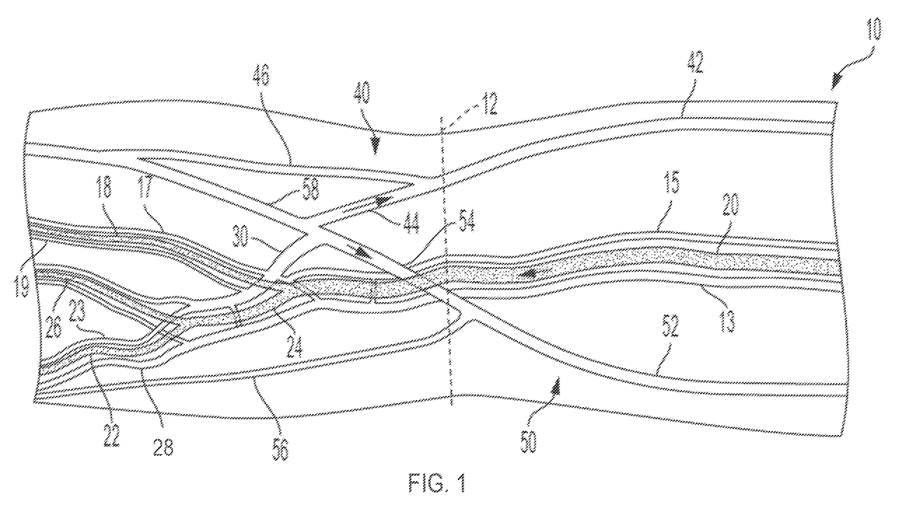

FIG. 1 illustrates a simplified depiction of the typical vascular anatomy an arm 10 around an elbow joint 12 including one or more blood vessels that may be targeted for vascular treatment. As shown, the brachial artery 20 extends superficially and distally from the upper arm and sinks deeply into the arm near the elbow joint 12, where the brachial artery 20 branches into the radial artery 18 and the ulnar artery 24. The upper portion of the ulnar artery 24 is deeply seated within the arm 10 beneath the superficial flexor muscles (not shown), and leads down the ulnar side of the forearm to the wrist. Further down the arm 10, typically just below the radial tuberosity of the radius bone (not shown), the ulnar artery 24 branches into the interosseous artery 26 and the deep ulnar artery 22. The interosseous artery 26 eventually feeds into the posterior and anterior interosseous arteries (not shown).

Also shown in FIG. 1 are the cephalic vein 40 and the basilic vein 50. The cephalic vein 40 includes the upper cephalic vein 42, the median cephalic vein 44, and the lower cephalic vein 46. The basilic vein 50 includes the upper basilic vein 52, the median basilic vein 54, and the lower basilic vein 56. The upper cephalic vein 42 runs along the outer border of the bicep muscle (not shown) and continues down into the forearm as the lower cephalic vein 46. The median cephalic vein 44 joins the upper cephalic vein 42 near the elbow joint 12. The upper basilic vein 52 runs along the inner side of the bicep muscle (not shown) and continues into the forearm as the lower basilic vein 56. The lower basilic vein 56 of the lower arm is sometimes referred to as the common ulnar vein. The median basilic vein 54 (in some instances referred to as the median cubital vein) joins the upper basilic vein 52 and the lower basilic vein 56. The median basilic vein 54 and the median cephalic vein 44 are formed at the branching of the median antebrachial vein 58. Near the branching of the median antebrachial vein 58 into the median basilic vein 54 and the median cephalic vein 44, a perforating branch 30 connects these vessels with the deep veins of the arm through the antebrachial fascia (not shown).

As shown in FIG. 1, perforating branch 30 communicates with a first deep ulnar vein 23 and a second deep ulnar vein 28. These deep ulnar veins 23/28 may run substantially parallel on either side of the deep ulnar artery 22. The deep ulnar artery 22 may branch away from ulnar artery 24 distal to the interosseous artery 16. Between the brachial artery 20 and the interosseous artery 26, the deep ulnar veins 23/28 are typically located in close proximity to the deep ulnar artery 22, and usually less than 2 mm separate the deep ulnar artery 22 from the deep ulnar veins 23/28. Along the length of the deep ulnar veins 23/28, transverse branches (not shown) may occasionally connect to the deep ulnar veins 23/28.

Also shown in FIG. 1 are first brachial vein 13 and second brachial vein 15. The brachial veins 13/15 generally run along the brachial artery 14, and the deep ulnar veins 23/24 feed into the brachial veins 13/15 near the elbow joint. Additionally, a pair of radial veins 17/19 may run along the radial artery 18, and may feed into one or both of the brachial veins 13/15.

In various embodiments, access to the ulnar artery and/or the ulnar vein may be achieved through an access site formed at the wrist or further up the arm into a superficial vein or artery. The catheter(s) may then be advanced through the vasculature to a treatment location. For example, it is often desirable to form a fistula between a vein and an artery proximate to a perforator (e.g., perforating branch 30) to increase blood flow from deep arteries to the superficial veins for such purposes as dialysis. Advancing a catheter from a superficial vein or artery makes accessing the site for fistula formation within the deep arterial/venous system easier.

It is noted that the vasculature within an arm is illustrated for example purposes only. It is contemplated that systems as described herein may be used to treat blood vessels anywhere within a body, human or animal (e.g., bovine, ovine, porcine, equine, etc.). For example, in some embodiments, blood vessels which are targeted and treated may include the femoral artery and femoral vein or the iliac artery and the iliac vein. In other embodiments, treatments between body conduits may not be limited to vein/artery treatments but may include treatment or fistula formation between adjacent veins, adjacent arteries or any other body conduits (e.g., bile ducts, esophagus, etc.).

### Catheters and Catheter Systems

Generally, systems described herein are directed to endovascular treatment of a blood vessel. For example, systems described herein may be useful in measuring, modifying, and/or ablating tissue to form a fistula. The systems described here typically include one or more catheters. The one or more catheters may comprise one or more treatment portions. The one or more treatment portions may include one or more fistula-forming elements that include one or more thermoelectric generators. The catheters described may further comprise elements to aid in fistula formation that is minimally invasive while providing precise and modulated temperature control, as described in more detail herein.

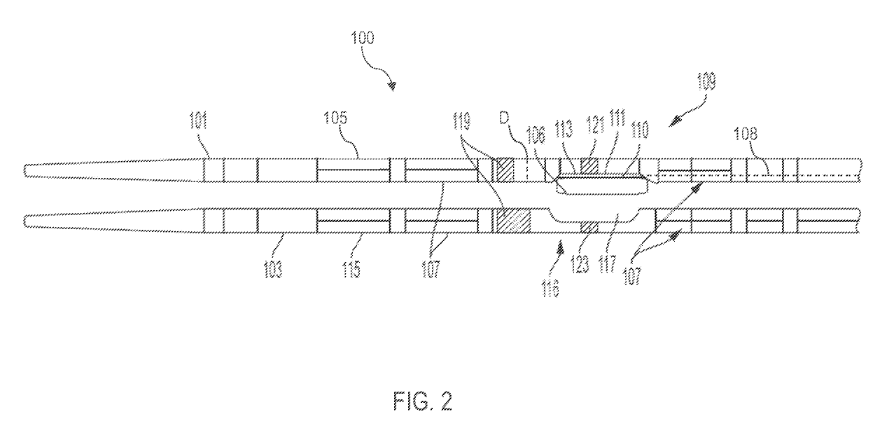

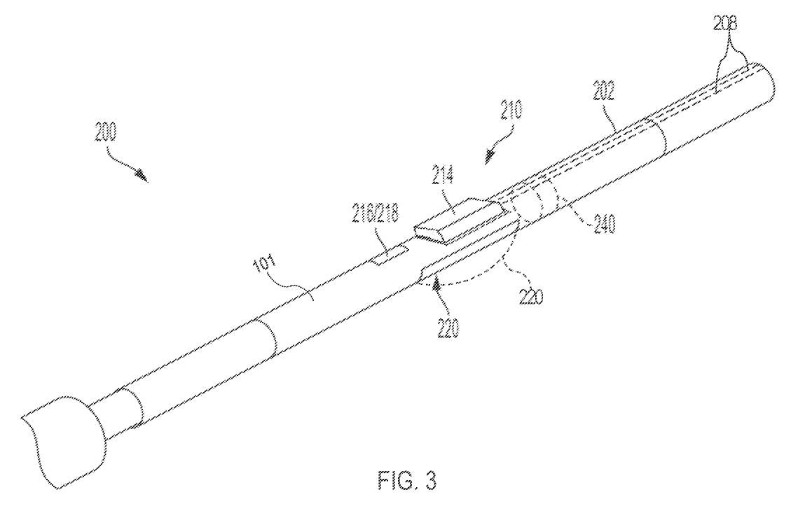

Referring now to FIGS. 2 and 3, various embodiments of one or more catheters are depicted. FIG. 2 generally illustrates one embodiment of a two catheter system 100. FIG. 3 illustrates an embodiment of a single catheter system 200. Accordingly, in embodiments incorporating a single catheter system 200, a second catheter may not be necessary for supplying a desired treatment to a blood vessel. However, it is noted that various features of either the two catheter system 100 or the single catheter system 200 may be incorporated into either of the two systems. For example, a thermoelectric generator such as illustrated in the single catheter system 200 may be the same as a thermoelectric generator used in the two catheter system 100.

FIG. 2 generally illustrates one embodiment of a two catheter system 100 configured to be used to form a fistula. As shown, the system may include a first catheter 101 and a second catheter 103. The first catheter 101 may comprise a catheter body 105, one or more magnetic elements 107, and a treatment portion 109. Similarly, second catheter 103 may comprise a catheter body 115, one or more magnetic elements 107, and a treatment portion 116. The first catheter 101, the second catheter 103, or both may have any suitable diameter for intravascular use, such as, for example, 4 French, 5.7 French, 6.1 French, 7 French, 8.3 French, between 4 French and 9 French, between 4 French and 7 French, between 4 French and 6 French, or the like.

As described herein, embodiments may be directed to fistula formation, and accordingly, the first catheter 101 may include a fistula-forming element 110 that may be used to form a fistula. The fistula-forming element 110 may comprise a thermoelectric generator 106, which will be described subsequently in more detail.

In some variations, the first catheter 101 may comprise a housing 113, which may help protect other components of the first catheter 101 during fistula formation. For example, when the fistula-forming element 110 comprises a thermoelectric generator 106 configured to ablate tissue, the housing 113 may comprise one or more insulating materials that may shield or otherwise protect one or more components of the first catheter 101 from heat that may be generated by the thermoelectric generator 106 during use. However, as will be described in greater detail below, the thermoelectric generator 106 is configured to provide a temperature differential, which may allow for elimination of or less insulating materials incorporated within the first catheter 101. That is, one or more insulating materials may not be required to shield or otherwise protect one or more components of the first catheter 101 from heat that may be generated by the thermoelectric generator 106 during use.

In some variations, the fistula-forming element 110 may be affixed to the housing 113 so that it projects out of an opening 111 in the catheter body 105 beyond an external diameter D of the catheter body 105. In some variations, the fistula-forming element 110 may be moveable so as to be advanced to project out of the opening 111 in the catheter body 105 and/or retracted into the opening 111. For example, the fistula-forming element 110 may be configured to move between a low-profile configuration and an extended configuration in which it extends from the catheter body 105. In such embodiments, the fistula-forming element 110 may be held in the low-profile configuration during placement of the first catheter 101. For example, in some variations the fistula-forming element 110 may be held in the low-profile configuration by the catheter body 105 or by a sleeve (not shown) advanceable and retractable over the fistula-forming element 110. The fistula-forming element 110 may be released from the low-profile configuration when the fistula-forming element 110 has been delivered to the location for fistula formation.

In some variations the fistula-forming element 110 may be spring-biased toward the extended configuration. That is, the fistula-forming element 110 may be configured to self-expand from the low-profile configuration to the extended configuration. Put yet another way, the fistula-forming element 110 may be in its natural resting state in the extended configuration.

Referring back to FIG. 2, it should be appreciated that the catheters of the systems described herein may comprise magnetic elements 107 including one or more magnets; and each catheter may comprise any number of individual magnets (e.g., one, two, three, four, five, six, seven, or eight or more, etc.). In this way, when the catheters of the systems described herein are brought together, the attractive magnetic forces of the magnets may bring the catheters and blood vessels in closer approximation. In variations in which a catheter comprises a plurality of magnets, these magnets may be grouped into one or more magnet arrays. The magnets may be located inside and/or outside of a catheter body. The magnets may be positioned at any suitable location along the length of the catheter. Generally, the dimensions of the magnets described herein may be selected based on the size of the catheters carrying the magnets, which in turn may be selected based on the anatomical dimensions of the vessels through which the catheters may be advanced. For example, if the catheter is to be advanced through a blood vessel having an internal diameter of about 3 mm, it may be desirable to configure any magnet to be less than about 3 mm at the widest part of its cross-section, to reduce the risk of injury to vessel walls during advancement and manipulation of the catheter. Each magnet may have any suitable length (e.g., about 5 mm, about 10 mm, about 15 mm, about 20 mm, or the like), although it should be appreciated that in some instances longer magnets may limit the flexibility of the catheter to maneuver through tissue.

The magnets elements 107 may be permanent magnets comprising one or more hard magnetic materials, such as but not limited to alloys of rare earth elements (e.g., samarium-cobalt magnets or neodymium magnets, such as N52 magnets) or alnico. In some variations, the magnets may comprise anisotropic magnets; in other variations, the magnets may comprise isotropic magnetics. In some variations, the magnets may be formed from compressed powder. In some variations, a portion of the magnets (e.g., a permeable backing) may comprise one or more soft magnetic materials, such as but not limited to iron, cobalt, nickel, or ferrite.

When the magnets are located within the catheter, as in FIG. 2 for example, given the limitations on magnet size, it may be desirable in some instances to use magnets configured to produce magnetic fields that increase the magnetic force that can be generated with a magnet of a given size. For example, in some variations the system may comprise one or more of the magnets described in U.S. Pat. Application Serial No. 14/214,503, filed on Mar. 14, 2014, and titled “FISTULA FORMULATION DEVICES AND METHODS THEREFOR,” and/or U.S. Pat. Application Serial No. 14/657,997, filed on Mar. 13, 2015, and titled “FISTULA FORMATION DEVICES AND METHODS THEREFOR,” both of which are hereby incorporated by reference in their entireties.

It should be appreciated that while some of the systems described here comprise a first catheter 101 and a second catheter 103 each comprising one or more permanent magnets, in other variations either the first catheter 101 or second catheter 103 may comprise ferromagnetic elements (i.e., elements attracted to but not generating a permanent magnetic field). For example, in some variations, the first catheter 101 may include only one or more ferromagnetic elements while the second catheter 103 comprises one or more permanent magnets. In other variations, the second catheter 103 may include only one or more ferromagnetic elements while the first catheter 101 comprises one or more permanent magnets. However, in other variations, one or both of the first and second catheters 101/103 may include any suitable combination of ferromagnetic, permanent, and/or other suitable kinds of magnets.

Referring still to FIG. 2, the second catheter 103 may comprise a catheter body 115 and one or more magnetic elements 107. In variations where the first catheter 101 comprises a fistula-forming element 110 configured to project out the catheter body 105 of the first catheter 101, such as the variation depicted in FIG. 2, the catheter body 115 of the second catheter 103 may comprise a treatment portion 116 that includes a recess 117 formed therein, which may be configured to receive the fistula-forming element 110 as it passes through tissue. While shown in FIG. 2 as having a recess 117, it should also be appreciated that in some variations, the treatment portion 116 of the second catheter 103 may not include a recess 117.

In embodiments, the recess 117 may be coated by an insulating material (not shown), which may act as a backstop to receive and contact the thermoelectric generator 106 of the first catheter without damaging one or more components of the first catheter 101. As will be described subsequently in more detail, the exposed surface of the thermoelectric generator 106, that is, the surface exposed outside of the housing 113, may be heated to a temperature greater than a concealed surface of the thermoelectric generator 106. The heating may occur by applying an electric current to one of the exposed surface and the concealed surface thereby producing a temperature differential that heats the exposed surface of the thermoelectric generator 106. In embodiments, the electric current may be reversed, thereby cooling the exposed surface of the thermoelectric generator 106 and heating the concealed surface of the thermoelectric generator 106. Therefore, because the thermoelectric generator 106 may be utilized to cool one or both of the exposed surface and the concealed surface, the incorporation of the thermoelectric generator 106 may thereby eliminate the need for coating an insulating material on the recess 117.

In some variations, the treatment portion 116 of the second catheter 103 may include a fistula-forming element (not shown) in addition to or instead of the fistula-forming element 110 of the first catheter 101. Thus, in some variations, a fistula may be formed by the thermoelectric generator 106 of one catheter, while in other variations, two catheters each comprising a thermoelectric module may simultaneously heat tissue from opposing sides to form a fistula. For example, a first catheter placed in a first vessel and a second catheter placed in the second catheter may be aligned such that a thermoelectric module of the first catheter is aligned with the thermoelectric module of the second catheter and tissue of the first vessel and the second vessel is sandwiched between the thermoelectric modules. Energization of the thermoelectric modules may cause the tissue to weld together and ablate to create an opening between the first vessel and the second vessel.

Referring still to FIG. 2, in variations, each of the one or more catheters may include one or more location indicators 119 configured to provide location information of the one or more catheters to allow a control unit of the system to determine a location of the treatment portion of the catheter as it is advanced through the vascular of a subject (e.g., patient). For example, in one embodiment, each of the first catheter 101 and the second catheter 103 may include echogenic markers. The echogenic markers may be positioned proximate to the treatment portion of the catheter and may be visible to an imaging device, such as an ultrasound imaging device. The echogenic markers may form particular patterns (e.g., a series of different sized echogenic rings with a specific spacing similar to a bar code), which may allow recognition of a particular catheter. Such pattern or ring may include marker bands made from, for example, platinum, iridium, or combinations thereof applied to the catheter proximate to the treatment portion of the catheter. In some embodiments, a control unit, using an imaging device to capture image data of the one or more catheters, may be configured to determine a location of the treatment portion of the one or more catheters based on the echogenic markers. In a two-catheter system such as illustrated in FIG. 2, each of the first and second catheters 101/103 may include echogenic markers which may be identical to or different from one another. Where the echogenic markers on each of the first and second catheters 101/103 vary from one another, a control unit may be able to determine which catheter is which. In some embodiments, echogenic markers may be used to indicate a rotational orientation of the one or more catheters. For example, a pattern of the echogenic marker when viewed under ultrasound may indicate in which direction the treatment portion of the particular catheter is facing.

In embodiments, in addition to or in lieu of echogenic markers, the catheters 101/103 may include one or more location sensors 121/123, configured to output a signal indicative of a location of the catheter 101/103 (e.g., the treatment portion of the catheter). For example, the location sensor 121/123 may include an active electromagnetic sensor, a passive electromagnetic sensor, a permanent magnet, an RFID device, and or/ an ultrasound transceiver. In embodiments, a control unit may, based on the signal received from the location sensor 121/123, determine a location of the treatment portion 109/116 of the catheter 101/103 and follow a location of the catheter 101/103 in real time with an imaging device. The location sensor 121/123 may be coupled to or positioned within the housing 113 of the catheter 101/103. For example, a location sensor 121/123 may be positioned longitudinally within the treatment portion 109/116 of the catheter 101/103. In some embodiments, a location sensor may be positioned proximal to and/or distal from the treatment portion 109/116 of the catheter 101/103. It is noted that while the one or more location sensors 121/123 are illustrated as being in close proximity to the treatment portion 109/116, the one or more location sensors may be positioned anywhere along the housing of the catheter 101/103.

Referring to FIG. 3, an embodiment of a single-catheter system 200 including a catheter 201 is shown. Catheter 201 of the single catheter system 200 may be substantially similar to the first catheter 101 of the two catheter system described above. Similar to the first catheter 101 described in regards to FIG. 2 above, the catheter 201 may include a housing 202. In embodiments, a treatment portion 210 may be coupled to the housing 202.

In embodiments wherein endovascular treatment is directed to fistula formation, the treatment portion 210 may include a fistula-forming element 214 or other cutting device for forming a fistula. While the illustrated embodiment depicts the fistula-forming element 214 as a plate, the fistula-forming element 214 may be substantially similar to the fistula-forming element 110 described above, which may comprise a thermoelectric generator 106 described subsequently in more detail.

It is also contemplated that the catheter 201 may include one or more echogenic markers 216 and/or one or more location sensors 218, as described above in regard to FIG. 1.

In embodiments, the catheter 201 may include one or more biasing mechanisms 220. The one or more biasing mechanisms 220 may be configured contact a wall of a blood vessel to bias the treatment portion 210 of the catheter 201 into contact with the wall (e.g., at a target treatment location) of the blood vessel. That is, the one or more biasing mechanisms 220 may expand away from the body of the catheter (as illustrated in FIG. 3) to cause the catheter 201 to move laterally within the host blood vessel to cause the treatment portion 210 (e.g., thermoelectric module) to contact a wall of the blood vessel. In some embodiments, the force of the one or more biasing mechanisms 220 may alter a shape of the blood vessel to extend the blood vessel in a direction opposite the movement of the biasing mechanism. Accordingly, the one or more biasing mechanisms 220 may be any mechanism configured to move the catheter transversely within a blood vessel to cause the treatment portion of the catheter to contact a treatment location within the blood vessel. The one or more biasing mechanisms 220 may be positioned on opposite sides of the housing 202 from the treatment portion 210 of the catheter 201. In embodiments, the one or more biasing mechanisms 220 may include, but are not limited to, balloons, cages, expandable wires, other expandable/retractable mechanisms, etc.

Embodiments of the thermoelectric generator (TEG) will now be described for use in either the two catheter system 100 or the single catheter system 200. As stated previously, a thermoelectric generator (TEG), which may also be referred as an electrothermal module (ETM), a thermoelectric module, or a Seebeck generator, is a solid state device that converts electrical energy into a temperature differential across the thermoelectric generator. Thermoelectric generators may also convert temperature differences directly into electrical energy (e.g., though a Seebeck effect phenomenon in which a temperature differential between two electrically-connected junctions produces an electromagnetic force between the junctions). Thermoelectric generators may also operate such that applying a voltage to the device can cause it to act as a heater or cooler, depending on the magnitude and polarity of the voltage (e.g., though a Peltier effect phenomenon in which voltage applied across two electrically-connected junctions produces a temperature differential between the junctions).

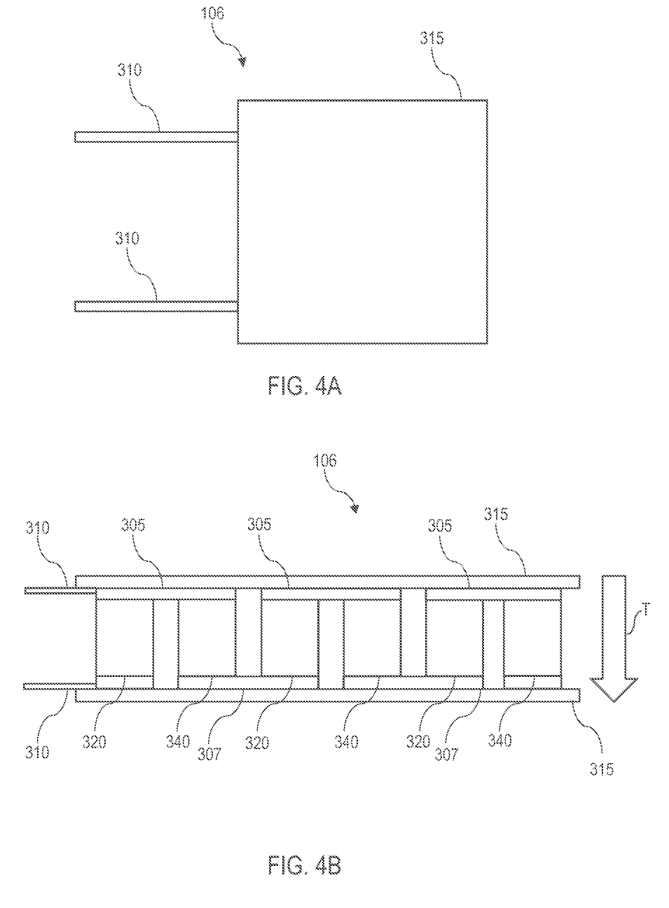

Referring now to FIGS. 4A-4B, in embodiments, the thermoelectric generator 106 may include thermoelectric materials 305/307. As used herein, a “thermoelectric material” may be defined as a material that responds to electrical potential by creating a temperature differential. Suitable thermoelectric materials may have both relatively high electrical conductivity and relatively low thermal conductivity. Having a low thermal conductivity may ensure that when one side of the thermoelectric generator is heated, the other side is cooled. For example, the thermoelectric material may include semiconductor materials. In some embodiments, the thermoelectric materials may include one or more alloys based on bismuth (Bi), antimony (Sb), tellurium (Te), selenium (Se), lead (Pb). In further embodiments, the thermoelectric material may include one or more of bismuth telluride (Bi₂Te₃), lead telluride (PbTe), and silicon germanium (SiGe).

The thermoelectric generator 106 may include an electrical circuit comprising the thermoelectric materials 305/307. In embodiments, the thermoelectric generator 106 may include dissimilar thermoelectric materials 305/307 on opposite sides of the thermoelectric generator 106. As used herein “dissimilar thermoelectric materials” refers to thermoelectric materials having different thermoelectric properties from each other. In embodiments, the thermoelectric generator 106 may include dissimilar thermoelectric materials 305/307 thermally in parallel. In some embodiments, the thermoelectric modules may include dissimilar thermoelectric materials 305/307 electrically in series. In further embodiments, the thermoelectric module may include dissimilar thermoelectric materials 305/307 that are thermally in parallel and electrically in series.

Referring still to FIGS. 4A-4B, the dissimilar thermoelectric materials 305/307 may be in contact with plates 315. In embodiments, the plates 315 may be ceramic and referred to a first ceramic plate and a second ceramic plate. In embodiments, the types of plates 315 and thermoelectric materials 305/307 utilized may be selected based on the mechanical and thermal conditions of the treatment procedure performed in the body. For example, the thermoelectric module may be subject to thermally-induced stresses and strains as a result of the specific temperature gradient that may need to be applied to weld vessel and/or form a fistula between two vessels. Additionally, the thermoelectric modules may experience mechanical fatigue depending on the number of thermal cycles required for a particular treatment.

A temperature differential, indicated by arrow T, created between the dissimilar thermoelectric materials 305/307, joined by an n-type semiconductor 320 and a p-type semiconductor 340, may be caused by an electric current flowing through the circuit. An n-type semiconductor 320 may be a semiconductor having negative charge carriers. A p-type semiconductor 340 may be a semiconductor having positive charge carriers. The electric current may be produced by an energy source coupled to the thermoelectric generator 106 by wires 310. In embodiments, the magnitude of the current may be directly proportional to the temperature difference between the dissimilar thermoelectric materials 305/307. The temperature difference may cause one of the plates 315 to become heated while the other of the plates 315 is cooled.

In this way, the n-type semiconductor 320 and a p-type semiconductor 340 are configured so that a supplied electric current results in heating one of the plates 315 and cooling one of the plates 315 of the other side thermoelectric generator 106. A supplied second electric current opposite of the first electric current results in cooling of one of the plates 315 and heating one of the plates 315. For example, a supplied electric current may result in heating the plate 315 that is exposed outside of the housing of the catheter while cooling the plate 315 concealed within the housing of the catheter. A supplied second electric current opposite of the first electric current results in cooling the plate 315 that is exposed outside of the housing of the catheter while heating the plate 315 concealed within the housing of the catheter.

In embodiments, circuitry may be employed to control cycling and temperature ranges of the heating and the cooling. By way of example, and not as a limitation, one of the plates 315 may be heated to a temperature from 10° C. to 120° C. while the opposing plate is cooled, and other temperatures lower than 10° C. and above 120° C. are contemplated and possible. In embodiments, one of the plates 315 may be heated to a temperature from 10° C. to 100° C., from 10° C. to 80° C., from 10° C. to 50° C., from 10° C. to 30° C., from 30° C. to 120° C., from 30° C. to 100° C., from 30° C. to 80° C., from 30° C. to 50° C., from 50° C. to 120° C., from 50° C. to 100° C., from 50° C. to 80° C., from 80° C. to 120° C., from 80° C. to 100° C., or from 100° C. to 120° C., while the opposing plate is cooled. The temperature difference between the plate that is heated and the opposing plate may be at least 70° C., at least 60° C., at least 50° C., at least 40° C., at least 30° C., at least 20° C., or at least 10° C. Then, the electric current may be reversed to cause the first one of the plates 315 to cool to and the opposing plate to heat. In such embodiments, the opposing plate may be heated to a temperature from 10° C. to 120° C., from 10° C. to 100° C., from 10° C. to 80° C., from 10° C. to 50° C., from 10° C. to 30° C., from 30° C. to 120° C., from 30° C. to 100° C., from 30° C. to 80° C., from 30° C. to 50° C., from 50° C. to 120° C., from 50° C. to 100° C., from 50° C. to 80° C., from 80° C. to 120° C., from 80° C. to 100° C., or from 100° C. to 120° C.,. The electric current may then be reversed again to heat the first one of the plates 315 to a temperature from about 10° C. to about 120° C. while the opposing plate is cooled. In this way, the electric current may be reversed any number of times sufficient to provide the desired clinical outcome. The electric current reversals may occur with a frequency of a time sequence cycle as determined by the operator to provide the desired clinical outcome. In example embodiments, the electric current reversals may occur at a frequency of the time sequence cycle defined by a switching period of approximately 2 seconds between each electric current reversal. In some embodiments, because the plates 315 can have their heating and cooling reversed, the need for coating an insulating material on the recess 117 (of treatment portion 116 in FIG. 2) may be eliminated.

As noted above, when current is driven through the thermoelectric generator 106, one of the plates 315 may be a heated and provide controllable, ablative or cutting thermal energy to an intended tissue site (the “target tissue”). For example, to provide increased heat, the current driven through the thermoelectric generator 106 may be modulated (e.g., increased) to provide heat to ablate and/or weld tissue together. Accordingly, the thermoelectric generator 106 may provide endovascular fistula formation treatment that is minimally invasive while providing precise and modulated temperature control. By requiring no moving parts to create the temperature differential, thermoelectric generators may reduce the complexity of conventional two catheter systems. Additionally, because thermoelectric generators may not require any fluids for fuel or cooling, they are non-orientation dependent, which may be particularly advantageous during endovascular treatments within the tortuous vasculature of a subject.

### Systems and Methods

Various systems and methods will now be described including the various embodiments of the above-described catheters. It is noted that while only specific embodiments may be illustrated within the figures, the present system and methods may be applicable to any of the catheter systems described herein.

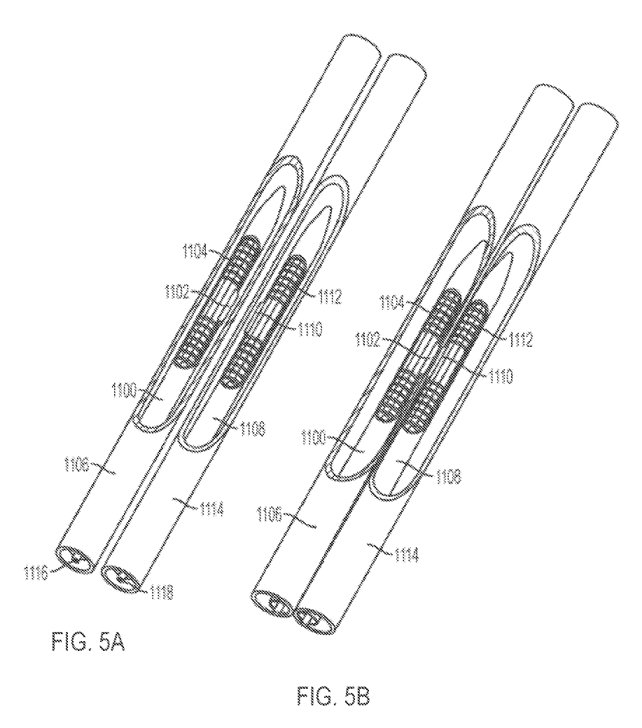

FIGS. 5A-5B illustrate a system in the vasculature comprising a first catheter 1100 in a first blood vessel 1106 and a second catheter 1108 in a second blood vessel 1114. The first catheter 1100 may be substantially similar to the first catheter 101 described above, unless otherwise noted. The second catheter 1108 may also be substantially similar to the second catheter 103 above, unless otherwise noted. In other embodiments, the system may only utilize one catheter. The first catheter 1100 may comprise a thermoelectric generator 1102, and the second catheter 1108 may comprise a thermoelectric generator 1102. The thermoelectric generators 1102/1110 may be connected via electrical leads 1116/1118 to a power source (not shown), as described in more detail herein.

The first catheter 1100 may further comprise one or more magnets 1104 that may be distal and proximal to the thermoelectric generator 1102. The second catheter 1108 may further comprise one or more magnets 1112 that may be distal and proximal to the thermoelectric generator 1102. Generally, the magnets may be configured to be attracted to one or more magnetic fields (e.g., produced by one or more magnets of the other catheter). The magnets may help to align or otherwise reposition the catheters 1100/1108 when placed in the vasculature. Once the first and second catheters 1100/1108 have been positioned, the attractive magnetic forces may also act to maintain the relative positions of the catheters 1100/1108. When the first and second catheters 1100/1108 are placed in respective blood vessels 1106/1114, tissue positioned between the blood vessels and/or limited compliance of the blood vessels may limit the extent to which the magnets of the first and second catheters bring the first and second catheters toward each other. The magnets may additionally or alternatively help to ensure that the catheters 1100/1108 are in proper axial and/or rotational alignment relative to each other. Such axial and /or rotational alignment of the catheters 1100/1108 may also facilitate alignment of the thermoelectric generators 1102/1110 relative to a target location for vessel adhesion.

The systems described herein may further comprise one or more additional alignment features to help ensure that the catheters are axially and/or rotationally aligned prior to heating the tissue. For example, one or both of the first and second catheters may comprise a visual alignment aid for indirectly or directly visualizing the alignment of a catheter within a tubular structure or relative to another catheter, such as via fluoroscopy, ultrasound, during positioning and/or alignment thereof.

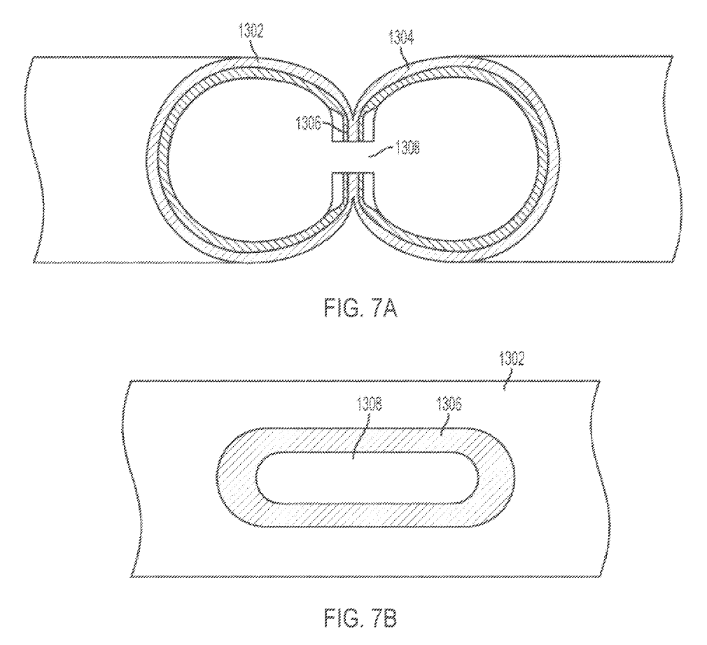

When the catheters 1100/1108 are brought together, the attractive magnetic forces of the magnets 1104/1112 may bring the catheters 1100/1108 and blood vessels 1106/1114 in closer approximation, as shown in FIG. 7B. One or more of the thermoelectric generators 1102/1110 may then be energized so as to apply heat to the tissue (e.g., through delivery of electrical current), as described in more detail hereinabove.

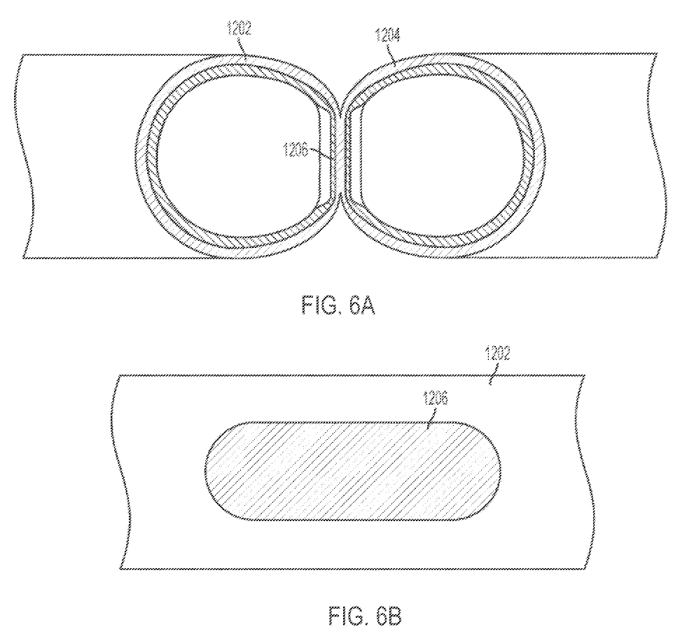

Once the catheters are aligned, one or more thermoelectric generators may be activated to adhere vessel tissue impinged between the first catheter 1100 and the second catheter 1108. As previously described, supplied electric current results in heating one of the plates of the thermoelectric generators 1102/1110, for example the plate exposed outside of the housing of the catheter while cooling the plate concealed within the housing of the catheter 1100/1108. As described with respect to FIG. 4B, a supplied second electric current opposite of the first electric current results in cooling of one of the plates 315 and heating one of the plates 315. As shown in FIG. 6A, the heat from the one or more thermoelectric generators may form a thermal weld 1206 between a first vessel 1202 and a second vessel 1204. FIG. 6B is a plan view of the first vessel 1202 having formed a thermal weld 1206 in the shape of the thermoelectric generators in contact with the first vessel 1202. In some instances, tissue may be heated to form a thermal weld between the intimal, medial, and/or adventitia of the vessels 1202/1204. The thermal weld 1206 may form a hermetic seal between the vessels, thereby preventing pressurized fluid from ingress or egress through the weld plane. The weld may also be strong enough to prevent the vessels from being pulled apart under forces that may be applied due to bodily function or motion. In other instances, the thermal weld 1206 may be able to withstand internal hydraulic pressure from dissecting the vessels apart. In some variations, the weld may have a width of about 0.1 mm to about 15 mm and a length ranging from about 0.1 mm to about 10 cm, although the weld length may vary from this range. In some variations, a plurality of discrete welds may be produced by a catheter system using a plurality of adhesion elements, or movement of the catheter(s) through the vessel(s).

As stated previously herein, the thermoelectric generators may adhere tissue by heating the tissue. That is, the thermoelectric generator(s) deliver thermal energy to adhere the tissue through the exposed surface of the thermoelectric generator being heated by driving a current through the thermoelectric generator(s). In some variations, the thermoelectric generators may heat tissue by delivering thermoelectric energy.

Referring now to FIGS. 7A-7B, in variations in which a fistula is formed between the two vessels after adhesion, the weld may maintain adhesion of the two attached vessels when the fistula is subsequently formed in the weld. In other words, a weld may prevent pressurized fluids traveling through the fistula from breaching the hermetic seal. In this way, the weld may prevent extravasation or leaking of fluids and thus may provide an enhanced fistula. As noted above, the current driven through the thermoelectric generator provide controllable, ablative or cutting thermal energy to an intended tissue site (the “target tissue”). To provide increased heat, the current driven through the thermoelectric generator may be modulated (e.g., increased) to provide sufficient heat to ablate the tissue. Accordingly, the thermoelectric generator may provide endovascular fistula formation treatment that is minimally invasive while providing precise and modulated temperature control. FIG. 7A shows a cross-sectional view of a thermal weld 1306 surrounding a fistula 1308 between a first vessel 1302 and a second vessel 1304. FIG. 7B shows a plan view of the first vessel 1302 and thermal weld 1306 and a fistula 1308 formed therethrough to provide fluid communication through the fistula 1308 while maintaining a perimeter of the welded tissue of thermal weld 1306 to prevent fluid leakage.

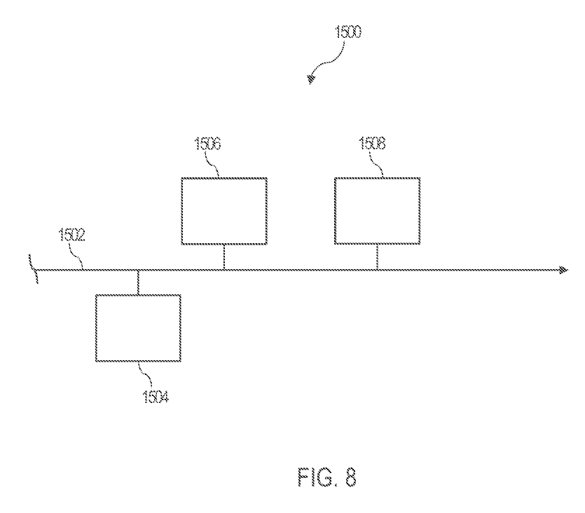

FIG. 8 generally schematically depicts communication between various modules within a system 1500 for endovascular treatment of a blood vessel. In particular, the system 1500 includes a communication path 1502, a control unit 1504, and an energy source 1506 communicatively coupled to the control unit 1504. It is noted that in various embodiments a fewer or greater number of modules, generally represented as module 1508, may be included within the system 1500 without departing from the scope of the present disclosure. For example, in embodiments, the system 1500 may include a fluoroscopic imaging device, and a display, or other modules. Additionally, the system 1500 includes one or more catheters such as any of the two catheter or single catheter systems described herein above. That is, the system may include a single catheter system configured to generate a fistula or deliver another type of vascular treatment to a target location within a vessel or a dual-catheter system configured to generate a fistula between the two catheters or deliver some other type of vascular treatment.

The various modules of the system 1500 may be communicatively coupled to one another over the communication path 1502. The communication path 1502 may be formed from any medium that is capable of transmitting a signal such as, for example, conductive wires, conductive traces, optical waveguides, or the like. Moreover, the communication path 1502 may be formed from a combination of mediums capable of transmitting signals. In some embodiments, the communication path 1502 includes a combination of conductive traces, conductive wires, connectors, and buses that cooperate to permit the transmission of electrical data signals between the various components of the components such as processors, memories, sensors, input devices, output devices, and communication devices. Additionally, it is noted that the term “signal” means a waveform (e.g., electrical, optical, magnetic, mechanical or electromagnetic), such as DC, AC, sinusoidal-wave, triangular-wave, square-wave, vibration, and the like, capable of traveling through a medium.

The energy source 1506 of system 1500 may be operatively coupled to the one more catheters (e.g., the thermoelectric generator(s)) via an electrical lead. The energy source 1506 may be a handheld energy source to provide energy to one or more thermoelectric modules of the treatment portion of the catheter, as described above. In other embodiments, the energy source 1506 may be mounted to a moveable cart (not shown). In embodiments, one or more user input devices may be used to input commands into the control unit 1504 to excite the energy source 1506 for fistula formation. In embodiments, the energy source is disposable with the catheter(s) such that the entire system is disposable, thus requiring no capital equipment.

The control unit 1504 can be any type of computing device and includes one or more processors and one or more memory modules. The one or more processors may include any device capable of executing machine-readable instructions stored on a non-transitory computer-readable medium, such as those stored on the one or more memory modules. Accordingly, each of the one or more processors may include a controller, an integrated circuit, a microchip, a computer, and/or any other computing device.

The one or more memory modules of the control unit 1504 are communicatively coupled to the one or more processors. The one or more memory modules may be configured as volatile and/or nonvolatile memory and, as such, may include random access memory (including SRAM, DRAM, and/or other types of RAM), flash memory, secure digital (SD) memory, registers, compact discs (CD), digital versatile discs (DVD), and/or other types of non-transitory computer-readable mediums. Depending on the particular embodiment, these non-transitory computer-readable mediums may reside within the control unit 1504 and/or external to the control unit 1504. The one or more memory modules may be configured to store logic (i.e., non-transitory machine readable instructions) that, when executed by the one or more processors, allow the control unit to perform various functions that will be described in greater detail below.

As noted above, the system 1500 may further includes a display communicatively coupled to the other modules of the system 1500 over the communication path 1502. The display may be any type of display configured to display image data from an imaging device, such as a fluoroscopic imaging device or an ultrasound imaging device. In some embodiments, the control unit 1504 may process image data and with the display, project indicators onto the image to indicate, for example, rotational alignment, longitudinal alignment, distance between blood vessels, blood vessel labels (artery, catheter, perforator, etc.), etc. In embodiments wherein the imaging device comprises Doppler functionality, the control unit may be configured to display Doppler information include flow rate, volume, vessel pressure, etc. In various embodiments, the control unit may display the treatment portion of the catheter in real time as the treatment portion is advanced through the vasculature of the patient.

In some embodiments, though not shown during vascular treatment, a guidewire having an integrated tracking sensor close to its tip may be inserted into the desired vein or artery and advanced to a target treatment location under guidance of the imaging device. The catheter may then be advanced to the target treatment location over the guidewire using the one or more location sensors as described herein, or the one or more echogenic markers or rings, the treatment portion of the catheter may be tracked and displayed using the display device in real time with or without the use of fluoroscopy.

As noted herein, devices and methods as provided herein may be used for purposes other than fistula formation. For example, the devices as provided herein may be used for arterializing purposes (e.g., arterializing a vein for ischemia in the leg), vessel occlusion, angioplasty, thrombectomy, atherectomy, crossing, drug coated balloon angioplasty, stenting (uncovered and covered), lytic therapy, etc. In addition, methods provided herein, may include multiple treatments and or multiple treatment sites.

It should now be understood that embodiments as described herein are directed the systems, methods, and catheters for endovascular treatment of a blood vessel. In particular, embodiments as described herein include thermoelectric modules that provide endovascular fistula formation treatment that is minimally invasive while providing precise and modulated temperature control. Moreover, embodiments described herein may allow for use of a single catheter for such treatment as fistula formation. Thus simplifying such procedures for operators and patients alike.

It is noted that the terms “substantially” and “about” may be utilized herein to represent the inherent degree of uncertainty that may be attributed to any quantitative comparison, value, measurement, or other representation. These terms are also utilized herein to represent the degree by which a quantitative representation may vary from a stated reference without resulting in a change in the basic function of the subject matter at issue.

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

1. A system comprising a catheter for forming a fistula between two blood vessels, the catheter comprising:
a housing;
a treatment portion coupled to the housing, the treatment portion comprising:
a thermoelectric generator comprising an exposed surface exposed outside of the housing and a concealed surface opposite and electrically connected to the exposed surface; wherein:
the thermoelectric generator is configured to produce a temperature differential between the exposed surface and the concealed surface when an electric current is applied to one of the exposed surface and the concealed surface thereby producing the temperature differential between the exposed surface and the concealed surface to weld the two blood vessels together.

2. The catheter of claim 1, further comprising an energy source coupled to the thermoelectric generator.

3. The catheter of claim 2, wherein the energy source is a handheld energy source coupled to the thermoelectric generator.

4. The catheter of claim 1, wherein the thermoelectric generator comprises at least one n-type semiconductor and at least one p-type semiconductor disposed between and electrically connected to the exposed surface and the concealed surface.

5. The catheter of claim 1, wherein the thermoelectric generator comprises a thermoelectric material.

6. The catheter of claim 1, further comprising one or more location indicators configured to provide location information of the treatment portion of the catheter as it is advanced through a subject.

7. The catheter of claim 1, further comprising one or more biasing mechanisms configured contact a wall of a blood vessel to bias the treatment portion of the catheter into contact with the wall of the blood vessel.

8. A system for forming a fistula between two blood vessels, the system comprising:
a first catheter configured to be received in a first vessel, wherein the first catheter comprises:
a housing and a treatment portion coupled to the housing, wherein the treatment portion comprises a thermoelectric generator comprising an exposed surface exposed outside of the housing and a concealed surface opposite and electrically connected to the exposed surface;
a second catheter configured to be received in a second vessel adjacent to the first vessel; and wherein:
the thermoelectric generator is configured to produce a temperature differential between the exposed surface and the concealed surface when an electric current is applied to one of the exposed surface and the concealed surface thereby producing the temperature differential between the exposed surface and the concealed surface to weld the two blood vessels together.

9. The system of claim 8, further comprising an energy source coupled to the thermoelectric generator.

10. The system of claim 8, wherein the energy source is a handheld energy source coupled to the thermoelectric generator.

11. The system of claim 8, wherein the thermoelectric generator comprises at least one n-type semiconductor and at least one p-type semiconductor disposed between and electrically connected to the exposed surface and the concealed surface.

12. The system of claim 8, wherein the second catheter comprises a second housing and a second treatment portion coupled to the second housing, wherein the second treatment portion comprises a second thermoelectric generator.

13. The system of claim 8, wherein the first catheter, the second catheter, or both comprise one or more location indicators configured to provide location information of the first catheter, the second catheter, or both as it is advanced through a subject.

14. The system of claim 8, wherein the thermoelectric generator comprises at least one n-type semiconductor and at least one p-type semiconductor disposed between and electrically connected to the exposed surface and the concealed surface.

15. A method of forming a fistula, the method comprising:
advancing a first catheter into a first vessel, wherein the first catheter comprises a housing and a treatment portion coupled to the housing, and wherein the treatment portion comprises a thermoelectric generator comprising an exposed surface exposed outside of the housing and a concealed surface opposite and electrically connected to the exposed surface;
advancing a second catheter comprising a second treatment portion into a second vessel, wherein the second vessel is adjacent to the first vessel; and
wherein the thermoelectric generator is configured to produce a temperature differential between the exposed surface and the concealed surface when an electric current is applied to one of the exposed surface and the concealed surface thereby producing the temperature differential between the exposed surface and the concealed surface wherein the exposed surface is heated to a temperature greater than the concealed surface to weld the first blood vessel to the second blood vessel.

16. The method of claim 15, further comprising applying an electric current to the thermoelectric generator to produce the temperature differential.

17. The method of claim 15, further comprising reversing the electric current to produce a temperature differential between the exposed surface and the concealed surface such that the exposed surface is cooled to a temperature lesser than the concealed surface.

18. The method of claim 15, wherein the first catheter further comprises a first magnet, and the second catheter comprises a second magnet, and wherein the magnets are configured to bring the first catheter and the second catheter in closer approximation.

19. The method of claim 18, further comprising aligning the first magnet and the second magnet to align the treatment portion of the first catheter and the second treatment portion second catheter.

20. The method of claim 19, further comprising applying an electric current to the thermoelectric generator to produce the temperature differential after aligning the first magnet and the second magnet.
